(19) United States
(12) Patent Application Publication
(10) Pub. No.: US 2025/0375159 A1
(43) Pub. Date: Dec. 11, 2025

(54) WEARABLE DEVICE FOR MONITORING BODY WATER, ELECTRONIC DEVICE, AND OPERATION METHOD THEREFOR

(71) Applicant: SAMSUNG ELECTRONICS CO., LTD., Suwon-si (KR)

(72) Inventors: Hyoungseon CHOI, Suwon-si (KR); Youngjae OH, Suwon-si (KR); Jinhong MIN, Suwon-si (KR); Taehan JEON, Suwon-si (KR); Joonho KIM, Suwon-si (KR); Taeseon KIM, Suwon-si (KR); Seoyoung YOON, Suwon-si (KR)

(73) Assignee: SAMSUNG ELECTRONICS CO., LTD., Suwon-si (KR)

(21) Appl. No.: 19/311,946

(22) Filed: Aug. 27, 2025

Related U.S. Application Data
(63) Continuation of application No. PCT/KR2024/002534, filed on Feb. 27, 2024.

(30) Foreign Application Priority Data
Feb. 27, 2023 (KR) 10-2023-0025830
Mar. 27, 2023 (KR) 10-2023-0039892

(51) Int. Cl. A61B 5/00; A61B 5/0205; A61B 5/026; A61B 5/0531

(52) U.S. Cl. CPC A61B 5/4875; A61B 5/0008; A61B 5/02055; A61B 5/0531; A61B 5/681; A61B 5/0261; A61B 2562/0271

(57) ABSTRACT

A wearable device according to one or more embodiments of the disclosure may comprise, a communication circuit, an output device, an electrical sensor, an optical sensor, a temperature sensor, at least one processor, and memory. The instructions, when executed by the at least one processor individually or collectively, cause the wearable device to, obtain a first biosignal, a second biosignal, and a third biosignal related to the temperature from the electrical sensor, the optical sensor and the temperature sensor, respectively, transmit first data related to the first biosignal, second data related to the second biosignal, and third data related to the third biosignal to an external electronic device, receive data related to a body water change of a body from the external electronic device, and output feedback related to the body water change to the output device.

### CROSS-REFERENCE TO RELATED APPLICATION(S)

This application is a continuation application of International Application No. PCT/KR2024/002534 filed on Feb. 27, 2024, which claims priority to Korean Patent Application No. 10-2023-0025830, filed on Feb. 2, 2023, and Korean Patent Application No. 10-2023-0039892, filed on Mar. 27, 2023, in the Korean Intellectual Property Office, the disclosures of which are herein incorporated by reference herein in their entireties.

### BACKGROUND

### 1. Field

The disclosure relates to a wearable device, an electronic device, and an operating method thereof for monitoring a change in body water.

### 2. Description of Related Art

Maintaining body water is essential for basic human health, and is necessary to ensure proper blood circulation, functioning metabolism, waste excretion, and body temperature regulation. In particular, when body water decreases by 2% or more, deterioration of cognitive ability and motor ability may occur and, when body water decreases by 6% or more, it may even lead to death in severe cases.

In the related art, techniques used to detect such changes in body water have involved: a dilution method using a radioactive isotope, a whole body composition analysis method utilizing a bioelectrical impedance analysis (BIA) method that measures and analyzes impedance in the body, or a sweat patch that collects and analyzes sweat from the skin. However, these methods take a long time to measure, and their accuracy is fleeting, making it difficult to continuously monitor the state of body water. Further, the sweat patch method may only be utilized during exercise, when a large amount of sweat is discharged.

Further, in the case of an exercise state, which is a main cause of changes in body water, body temperature may rise, causing an error in measuring the change in body water.

### SUMMARY

According to an aspect of the disclosure, a wearable device may include a communication circuit configured to transmit data or a signal to or from an external electronic device or receive data or a signal from the external device, an output device, an electrical sensor measuring a first biosignal related to an electrical characteristic, an optical sensor measuring a second biosignal related to an optical characteristic, a temperature sensor measuring a temperature, and at least one processor operatively connected to the communication circuit, the output device, the electrical sensor, the optical sensor, and the temperature sensor. The at least one processor may be configured to obtain, the first biosignal from the electrical sensor, the second biosignal from the optical sensor, and the third biosignal related to the temperature from the temperature sensor. The at least one processor may be configured to transmit, through the communication circuit, first data related to the obtained first biosignal, second data related to the obtained second biosignal, and third data related to the third biosignal to the external electronic device. The at least one processor may be configured to receive, through the communication circuit, data related to a body water change of a body determined based on changes in the first data and the second data respectively corrected by the third data from the external electronic device. The at least one processor may be configured to output feedback related to the body water change to the output device based on the received data related to the body water change.

According to an aspect method of operating the wearable device according to an embodiment of the disclosure may include obtaining, a first biosignal related to an electrical characteristic from an electrical sensor, a second biosignal related to an optical characteristic from an optical sensor, and a third biosignal related to temperature from a temperature sensor. The method of operating the wearable device according to an embodiment may include transmitting, through a communication circuit, first data related to the obtained first biosignal, second data related to the obtained second biosignal, and third data related to the third biosignal to an external electronic device. The method of operating the wearable device according to an embodiment may include receiving, through the communication circuit, data related to a body water change of a body determined based on changes in the first data and the second data respectively corrected by the third data from the external electronic device. The method of operating the wearable device according to an embodiment may include outputting feedback related to the body water change to an output device based on the received data related to the body water change.

According to an aspect of the disclosure, a non-transitory, computer-readable storage medium storing one or more programs according to one or more embodiments of the disclosure may include obtaining, a first biosignal from an electrical sensor, a second biosignal from an optical sensor, and a third biosignal related to a temperature from a temperature sensor, based on execution of an application. The storage medium according to an embodiment may include transmitting, through a communication circuit, first data related to the obtained first biosignal, second data related to the obtained second biosignal, and third data related to the third biosignal to an external electronic device. The storage medium according to an embodiment may include receiving, through the communication circuit, data related to a body water change of a body determined based on changes in the first data and the second data respectively corrected by the third data from the external electronic device. The storage medium according to an embodiment may include outputting feedback related to the body water change to an output device based on the received data related to the body water change.

According to an aspect of the disclosure, an electronic device may include a communication circuit configured to transmit or receive data or a signal to an external device or receive data or a signal from the external device, an output device, and at least one processor operatively connected to the communication circuit and the output device. The at least one processor may be configured to obtain first data related to a first biosignal corresponding to an electrical characteristic, second data related to a second biosignal corresponding to an optical characteristic, and third data corresponding to a temperature. The at least one processor may be configured to correct the first data and the second data based on the third data. The at least one processor may be configured to determine data related to a body water change of a body based on changes in the corrected first data and the corrected second data. The at least one processor may be configured to transmit, through the communication circuit, the calculated data related to the body water change to the external device, or output feedback to the output device based on the calculated data related to the body water change.

According to an aspect of the disclosure, a method of operating an electronic device may include obtaining first data related to a first biosignal corresponding to an electrical characteristic, second data related to a second biosignal corresponding to an optical characteristic, and third data related to a temperature. The method of operating the electronic device according to an embodiment may include correcting the first data and the second data based on the third data. The method of operating the electronic device according to an embodiment may include determining data related to a body water change of a body based on changes in the corrected first data and the corrected second data. The method of operating the electronic device according to an embodiment may include transmitting, through a communication circuit, the calculated data related to the body water change to an external device, or outputting feedback to an output device based on the calculated data related to the body water change.

According to an aspect of the disclosure, a non-transitory, computer-readable storage medium storing one or more programs may include obtaining first data related to a first biosignal corresponding to an electrical characteristic, second data related to a second biosignal corresponding to an optical characteristic, and third data related to a temperature, based on execution of an application. The storage medium according to an embodiment may include correcting the first data and the second data based on the third data. The storage medium according to an embodiment may include calculating data related to a body water change of a body based on changes in the corrected first data and the corrected second data. The storage medium according to an embodiment may include transmitting, through a communication circuit, the calculated data related to the body water change to an external device, or outputting feedback to an output device based on the calculated data related to the body water change.

In a storage medium storing computer-readable instructions according to one or more embodiments of the disclosure, the instructions, when executed by at least one processor of a wearable device, may cause the wearable device to obtain the first biosignal, the second biosignal, and a third biosignal related to the temperature from an electrical sensor measuring a first biosignal related to an electrical characteristic, an optical sensor measuring a second biosignal related to an optical characteristic, and a temperature sensor measuring a temperature, respectively.

The instructions may, when executed by at least one processor of the wearable device, cause the wearable device to transmit, through a communication circuit configured to transmit or receive data or a signal with an external electronic device, first data related to the obtained first biosignal, second data related to the obtained second biosignal, and third data related to the third biosignal to the external electronic device.

The instructions may, when executed by at least one processor of the wearable device, cause the wearable device to receive, through the communication circuit, data related to a body water change of a body calculated based on changes in the first data and the second data respectively corrected by the third data from the external electronic device.

The instructions may, when executed by at least one processor of the wearable device, cause the wearable device to output feedback related to the body water change to an output device based on the received data related to the body water change.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram illustrating an electronic device in a network environment according to an embodiment of the disclosure;

FIG. 2A illustrates a block diagram illustrating a wearable device according to one or more embodiments of the disclosure;

FIG. 2B illustrates a connection state between a wearable device and an external electronic device according to one or more embodiments of the disclosure;

FIG. 3 is a flowchart regarding an operating method of a wearable device according to one or more embodiments of the disclosure;

FIG. 4 is a block diagram illustrating an electronic device according to one or more embodiments of the disclosure;

FIG. 5 is a flowchart regarding an operating method of an electronic device according to one or more embodiments of the disclosure;

FIG. 6 illustrates a change graph of body water calculated corresponding to whether temperature correction is performed according to one or more embodiments of the disclosure;

FIG. 7 illustrates a graph in which impedance of a plurality of frequencies changes according to a change in body temperature according to one or more embodiments of the disclosure;

FIG. 8A is a graph illustrating a change in a ratio of green light to infrared light of PPG (photoplethysmography) corresponding to whether temperature change occurs according to one or more embodiments of the disclosure;

FIG. 8B is a graph illustrating a change in PPG DC (direct current) level corresponding to whether temperature change occurs according to one or more embodiments of the disclosure;

FIG. 8C is a graph illustrating PPG waveforms corresponding to respective temperatures according to one or more embodiments of the disclosure;

FIG. 8D is a graph illustrating a change in PPG AC (alternating current) peak size corresponding to whether temperature change occurs according to one or more embodiments of the disclosure;

FIG. 9A illustrates a graph of a first weight designated according to a temperature of skin according to one or more embodiments of the disclosure;

FIG. 9B illustrates a graph of a second weight designated according to a temperature of skin according to one or more embodiments of the disclosure;

FIG. 10A is a graph illustrating a change in first data corresponding to respective circumstances of a user according to one or more embodiments of the disclosure;

FIG. 10B is a graph illustrating a change in second data corresponding to respective circumstances of a user according to one or more embodiments of the disclosure; and

FIG. 11 illustrates a second display of an electronic device providing visual feedback according to one or more embodiments of the disclosure.

### DETAILED DESCRIPTION

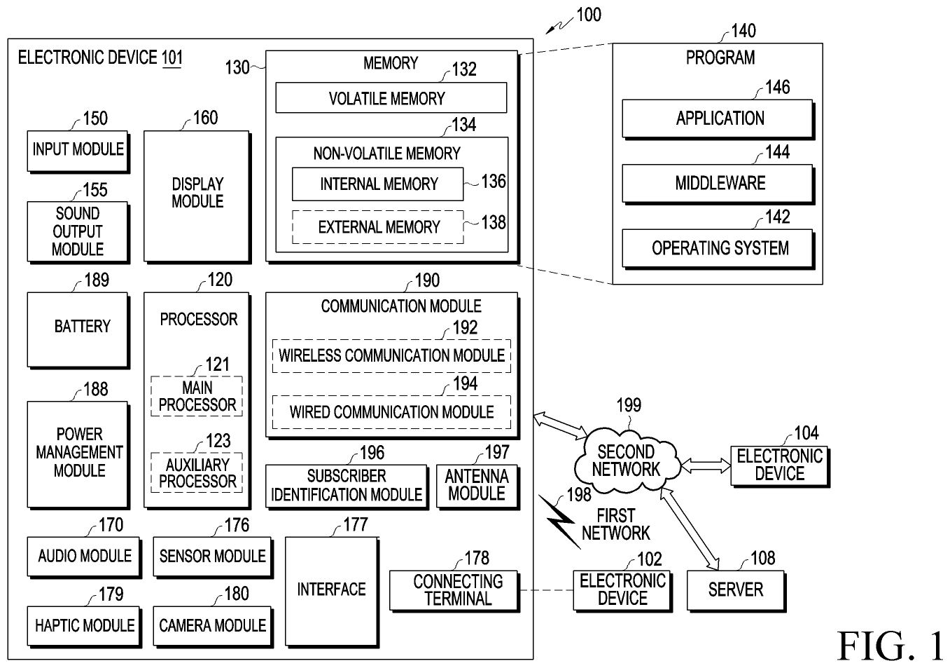

FIG. 1 is a block diagram illustrating an electronic device 101 in a network environment 100 according to an embodiment of the disclosure.

Referring to FIG. 1, the electronic device 101 in the network environment 100 may communicate with at least one of an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In an embodiment, at least one (e.g., the connecting terminal 178) of the components may be omitted from the electronic device 101, or one or more other components may be added in the electronic device 101. According to an embodiment, some (e.g., the sensor module 176, the camera module 180, or the antenna module 197) of the components may be integrated into a single component (e.g., the display module 160).

The processor 120 may execute, for example, software (e.g., the program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be configured to use lower power than the main processor 121 or to be specified for a designated function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. The artificial intelligence model may be generated via machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by other component (e.g., the processor 120) of the electronic device 101, from the outside (e.g., a user) of the electronic device 101. The input module 150 may include, for example, a microphone, a mouse, a keyboard, keys (e.g., buttons), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor configured to detect a touch, or a pressure sensor configured to measure the intensity of a force generated by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operation state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an accelerometer, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or motion) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via a first network 198 (e.g., a short-range communication network, such as Bluetooth™, wireless-fidelity (Wi-Fi) direct, or infrared data association (IrDA)) or a second network 199 (e.g., a long-range communication network, such as a legacy cellular network, a 5G network, a next-generation communication network, the Internet, or a computer network (e.g., local area network (LAN) or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device). According to an embodiment, the antenna module 197 may include one antenna including a radiator formed of a conductor or conductive pattern formed on a substrate (e.g., a printed circuit board (PCB)). According to an embodiment, the antenna module 197 may include a plurality of antennas (e.g., an antenna array). In this case, at least one antenna appropriate for a communication scheme used in a communication network, such as the first network 198 or the second network 199, may be selected from the plurality of antennas by, e.g., the communication module 190. The signal or the power may then be transmitted or received between the communication module 190 and the external electronic device via the selected at least one antenna. According to an embodiment, other parts (e.g., radio frequency integrated circuit (RFIC)) than the radiator may be further formed as part of the antenna module 197.

According to an embodiment, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. The external electronic devices 102 or 104 each may be a device of the same or a different type from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

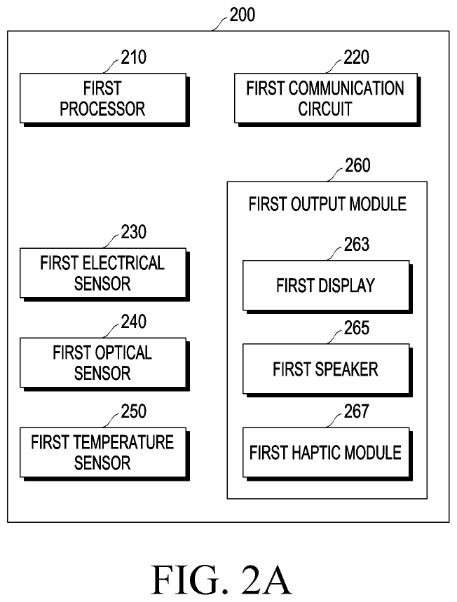

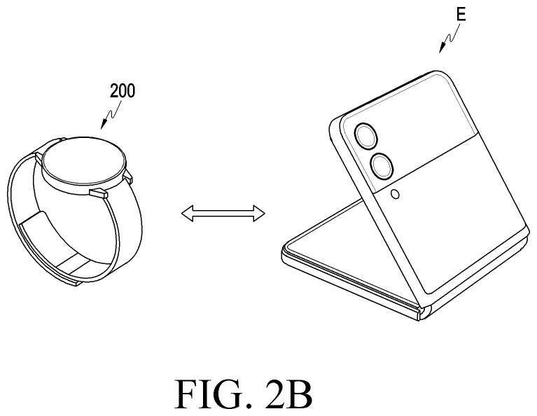

FIG. 2A illustrates a block diagram illustrating a wearable device 200 according to one or more embodiments of the disclosure. FIG. 2B illustrates a connection state between the wearable device 200 and an external electronic device E according to one or more embodiments of the disclosure.

Referring to FIG. 2A and FIG. 2B, a wearable device 200 (e.g., the electronic device 101 of FIG. 1) according to one or more embodiments may be a wearable type device that is bound to or mounted on a portion of a user's body. In one or more embodiments, the wearable device 200 may be a smart watch bound to the user's wrist, a head-mounted device (HMD) fixed to the user's head, or an ear-bud worn on the user's ear. In one or more embodiments, an external electronic device E (e.g., the external electronic device 102 of FIG. 1) may be a device that is communicatively connected to wirelessly transmit/receive signals or data with the wearable device 200. In one or more embodiments, the external electronic device E may be a portable communication device (e.g., a smartphone), a computer device, a portable multimedia device, or a portable medical device.

The wearable device 200 according to one or more embodiments may include a first processor 210 (e.g., the processor 120 of FIG. 1), a first communication circuit 220 (e.g., the communication module 190 of FIG. 1), a first electrical sensor 230, a first optical sensor 240, a first temperature sensor 250, and/or a first output module or device 260 (e.g., an output device such as the sensor module 176 of FIG. 1). In one or more embodiments, the wearable device 200 may include only some of the components illustrated in FIG. 2A, or may include additional components of the electronic device 101 illustrated in FIG. 1 that are not illustrated in FIG. 2A.

The first processor 210 according to one or more embodiments may perform an operation according to a command stored in first memory (e.g., the memory 130 of FIG. 1), or may perform an operation according to a command received from the external electronic device E. In one or more embodiments, the first memory may store an application, data, and/or operation commands corresponding to operations performed by the first processor 210.

The first communication circuit 220 according to one or more embodiments may be wirelessly communicatively connected to the external electronic device E. For example, a communication method may be implemented in various ways such as Bluetooth, BLE (Bluetooth Low Energy), or Wi-Fi. In one or more embodiments, the first communication circuit 220 may set a connection state with the external electronic device E through a designated communication method, or transmit and/or receive signals or data with the external electronic device E.

The wearable device 200 according to one or more embodiments may obtain biometric data of the user through the first electrical sensor 230, the first optical sensor 240, and/or the first temperature sensor 250.

The first electrical sensor 230 according to one or more embodiments may obtain a biosignal related to electrical characteristics. In one or more embodiments, an electrode electrically connected to the first electrical sensor 230 may contact the user's body. In one or more embodiments, the wearable device 200 or the first processor 210 may estimate biometric data such as the user's heart rate, HRV (heart rate variability), ECG (electrocardiogram), body temperature, water amount, sweat discharge amount, stress state, and breathing state using the biosignal obtained through the first electrical sensor 230.

For example, the first electrical sensor 230 may include an impedance sensor that measures data related to impedance corresponding to at least one frequency within a designated frequency band (e.g., about 1 [kHz] to about 3 [MHz] frequency band) range. For example, the first electrical sensor 230 may be an electrical dermal activity (EDA) sensor that measures galvanic skin response (GSR) or electrical dermal activity (EDA) related to electrical characteristics.

The first optical sensor 240 according to one or more embodiments may obtain a biosignal related to optical characteristics. In one or more embodiments, the first optical sensor 240 may obtain a biosignal related to optical characteristics by irradiating light onto the user's skin and obtaining light reflected by the skin. In one or more embodiments, the wearable device 200 or the first processor 210 may estimate biometric data such as the user's heart rate, respiratory rate, or blood pressure using the biosignal obtained through the first optical sensor 240.

For example, the first optical sensor 240 may be a photoplethysmogram (PPG) type optical blood flow measurement sensor (photoplethysmography, PPG) that measures pulse waves using light.

In one or more embodiments, the wearable device 200 or the first processor 210 may estimate biometric data such as the user's heart rate, HRV, ECG, body temperature, water amount, sweat discharge amount, stress state, and breathing state using the biosignal obtained through the first optical sensor 240.

The first temperature sensor 250 according to one or more embodiments may measure the user's body temperature. For example, the first temperature sensor 250 may measure the temperature of skin while in contact with the user's skin. In one or more embodiments, the first temperature sensor 250 may measure the user's body temperature using optical or other methods. The temperature may further comprise measured, estimated, and/or calculated temperatures and may further relate to, for example, core temperature, centroid temperature, deep-tissue temperatures, and/or other temperatures associated with any part of the body.

The first output module 260 according to one or more embodiments may output visual, auditory, and/or tactile feedback to the user. In one or more embodiments, the first output module 260 may include a first display 263 providing visual feedback, a first speaker 265 providing auditory feedback, and/or a first haptic module 267 providing tactile feedback.

In one or more embodiments, the first output module 260 may output feedback related to body water change. For example, the first display 263 may display visual data related to body water change on a screen. For example, the first speaker 265 may generate sound related to body water change. For example, the first haptic module 267 may generate vibration related to body water change.

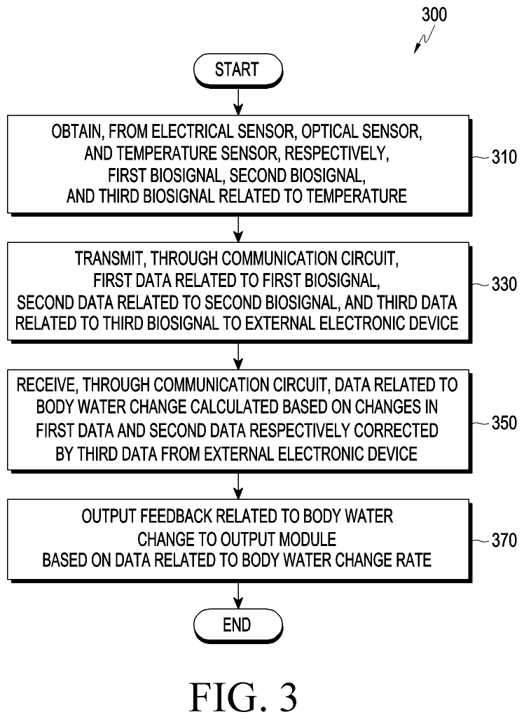

FIG. 3 is a flowchart 300 regarding an operating method of the wearable device 200 according to one or more embodiments of the disclosure.

Referring to FIG. 3, the wearable device (e.g., the wearable device 200 or the first processor 210 of FIG. 2A) according to one or more embodiments may obtain a first biosignal, a second biosignal, and a third biosignal related to temperature from an electrical sensor (e.g., the first electrical sensor 230 of FIG. 2A), an optical sensor (e.g., the first optical sensor 240 of FIG. 2A), and a temperature sensor (e.g., the first temperature sensor 250 of FIG. 2A), respectively, in operation 310.

In one or more embodiments, the wearable device 200 may obtain the first biosignal related to electrical characteristics from the electrical sensor 230. In one or more embodiments, the wearable device 200 may obtain the second biosignal related to optical characteristics from the optical sensor 240. In one or more embodiments, the wearable device 200 may obtain the third biosignal related to temperature from the temperature sensor 250, respectively.

In one or more embodiments, in operation 310, the wearable device 200 may obtain the first biosignal, the second biosignal, and/or the third biosignal simultaneously or sequentially, and the order of obtaining each signal may be changed in various ways.

The wearable device 200 according to one or more embodiments may transmit first data related to the obtained first biosignal, second data related to the obtained second biosignal, and third data related to the third biosignal to an external electronic device (e.g., the external electronic device E of FIG. 2B) through a communication circuit (e.g., the first communication circuit 220 of FIG. 2A) in operation 330.

In one or more embodiments, the wearable device 200 may set a communication connection corresponding to a designated communication method with the external electronic device E through the communication circuit 220. In one or more embodiments, the wearable device 200 may transmit the first data, the second data, and/or the third data to the external electronic device E in a designated communication method.

In one or more embodiments, the first data may be the first biosignal related to electrical characteristics obtained from the electrical sensor 230, and may be biometric data processed from the first biosignal. In one or more embodiments, the first data may be biometric data related to electrical characteristics such as impedance magnitude, impedance phase, resistance, reactance, or a ratio of high frequency to low frequency.

In one or more embodiments, the second data may be the second biosignal related to optical characteristics obtained from the optical sensor 240, and may be biometric data processed from the second biosignal. In one or more embodiments, the second data may be biometric data related to optical characteristics such as absorbance, PPG AC (alternating current) peak size, PPG DC (direct current) level, PPG DC to AC, DC low wavelength to DC high wavelength, AC low wavelength to AC high wavelength.

In one or more embodiments, the third data may be the third biosignal related to temperature obtained from the temperature sensor 250, and may be biometric data processed from the third biosignal.

In one or more embodiments, in operation 330, the wearable device 200 may transmit the first data, the second data, and/or the third data simultaneously or sequentially, and the order of transmitting each signal may be changed in various ways.

The wearable device 200 according to one or more embodiments may receive data related to a body water change of a body calculated based on changes in the first data and the second data respectively corrected by the third data from the external electronic device E through the communication circuit 220 in operation 350.

The external electronic device E according to one or more embodiments may calculate the body water change of the body based on the first data, the second data, and the third data as described below. In one or more embodiments, the external electronic device E may correct the first data and the second data, respectively, using the third data received from the wearable device 200, and calculate the body water change based on changes in the respectively corrected first data and second data. In one or more embodiments, the external electronic device E may transmit data related to the calculated body water change of the body to the wearable device 200.

The wearable device 200 according to one or more embodiments may receive data related to the body water change of the body calculated based on the transmitted first data, second data, and third data from the external electronic device E in response to transmitting the first data, the second data, and the third data to the external electronic device E.

In one or more embodiments, the body water change of the body may mean a change based on a designated specific body water state. For example, the body water change of the body may be a change rate (e.g., +2% or −3%) for a specific body water state. For example, the specific body water state may be a body water state in a state satisfying a designated time or designated condition, an average of accumulated measurements, or a designated value.

The wearable device 200 according to one or more embodiments may output feedback related to the body water change to an output module (e.g., an output device such as the first output module 260 of FIG. 2A) based on data related to the body water change received from the external electronic device E in operation 370.

The wearable device 200 according to one or more embodiments may display visual data related to the body water change on the first display 263. The wearable device 200 according to one or more embodiments may generate sound related to the body water change from the first speaker 265. The wearable device 200 according to one or more embodiments may generate vibration related to the body water change from the first haptic module 267.

The wearable device 200 according to one or more embodiments may output feedback corresponding to the body water change to the output module 260 in real-time based on data related to the body water change received from the external electronic device E. The wearable device 200 according to one or more embodiments may output feedback corresponding to the body water change to the output module 260 when the body water change received from the external electronic device E satisfies a designated condition.

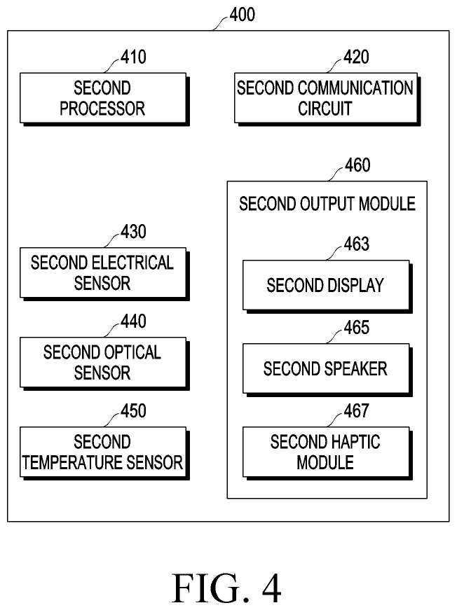

FIG. 4 is a block diagram illustrating an electronic device 400 according to one or more embodiments of the disclosure.

Referring to FIG. 4, an electronic device 400 (e.g., the electronic device 101 of FIG. 1) according to one or more embodiments may include a second processor 410 (e.g., the processor 120 of FIG. 1), a second communication circuit 420 (e.g., the communication module 190 of FIG. 1), a second electrical sensor 430, a second optical sensor 440, a second temperature sensor 450, and/or a second output module 460 (e.g., the sensor module 176 of FIG. 1). In one or more embodiments, the electronic device 400 may include only some of the components illustrated in FIG. 4, or may include additional components of the electronic device 101 illustrated in FIG. 1.

The electronic device 400 according to one or more embodiments may be a portable communication device (e.g., a smartphone), a computer device, a portable multimedia device, or a portable medical device. The electronic device 400 according to one or more embodiments may be a device that is communicatively connected to wirelessly transmit/receive signals or data with a wearable device (e.g., the wearable device 200 of FIG. 2A).

The electronic device 400 according to one or more embodiments may be a wearable type device such as a smart watch bound to the user's wrist that is bound to or mounted on a portion of the user's body, a head-mounted device (HMD) fixed to the user's head, or an ear-bud worn on the user's ear.

The second processor 410 according to one or more embodiments may perform an operation according to a command stored in second memory (e.g., the memory 130 of FIG. 1). In one or more embodiments, the second memory may store an application, data, and/or operation commands. Operations executed by the second processor 410 and/or operations stored in the second memory according to one or more embodiments is described below.

The second communication circuit 420 according to one or more embodiments may be wirelessly communicatively connected to the wearable device 200. For example, a communication method may be implemented in various ways such as Bluetooth, BLE, or Wi-Fi. In one or more embodiments, the second communication circuit 420 may set a connection state with the wearable device 200 through a designated communication method, or transmit and/or receive signals or data with the wearable device 200.

The electronic device 400 according to one or more embodiments may obtain biometric data of the user through the second electrical sensor 430, the second optical sensor 440, and/or the second temperature sensor 450. In one or more embodiments, the second electrical sensor 430, the second optical sensor 440, and/or the second temperature sensor 450 are components corresponding to the first electrical sensor 230, the first optical sensor 240, and/or the first temperature sensor 250, and descriptions related thereto may be omitted.

The electronic device 400 according to one or more embodiments may receive first data related to a first biosignal corresponding to electrical characteristics, second data related to a second biosignal corresponding to optical characteristics, and third data corresponding to a temperature of skin through the second communication circuit 420. For example, the wearable device 200 may measure the first biosignal, the second biosignal, and/or the third biosignal, and transmit the first data, the second data, and/or the third data to the electronic device 400 through a communication circuit (e.g., the first communication circuit 220 of FIG. 2A).

The second output module 460 according to one or more embodiments may output visual, auditory, and/or tactile feedback to the user. In one or more embodiments, the second output module 460 may include a second display 463 providing visual feedback, a second speaker 465 providing auditory feedback, and/or a second haptic module 467 providing tactile feedback. In one or more embodiments, the second output module 460 is a component corresponding to the first output module (e.g., the first output module 260 of FIG. 2A), and descriptions related thereto are omitted.

The electronic device 400 according to one or more embodiments may transmit data related to body water change to the wearable device 200 through the second communication circuit 420. For example, the wearable device 200 may receive data related to body water change through a communication circuit (e.g., the first communication circuit 220 of FIG. 2A). For example, the wearable device 200 may output visual, auditory, and/or tactile feedback to an output module (e.g., the first output module 260 of FIG. 2A) based on the received data related to body water change.

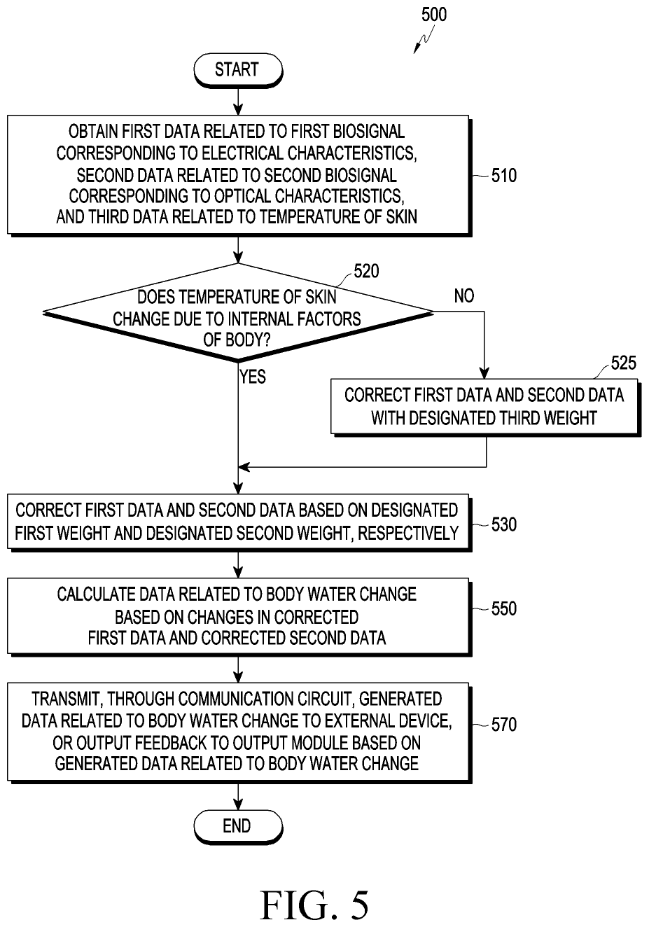

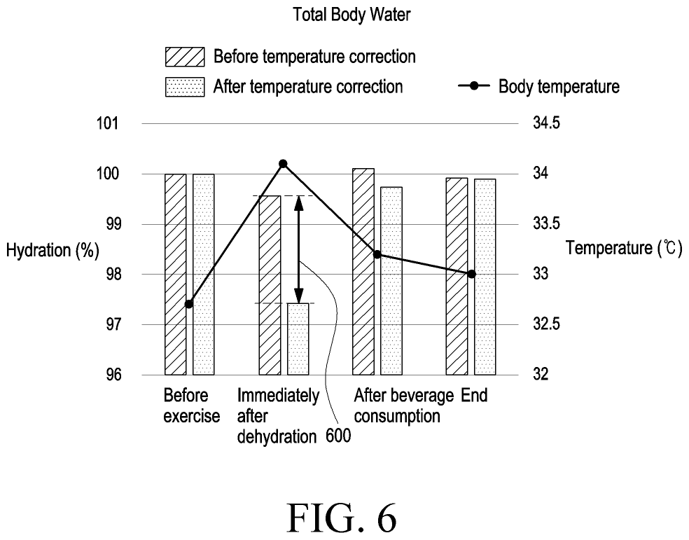

FIG. 5 is a flowchart 500 regarding an operating method of the electronic device 400 according to one or more embodiments of the disclosure. FIG. 6 illustrates a change graph of body water calculated corresponding to whether temperature correction is performed according to one or more embodiments of the disclosure.

Referring to FIG. 5, an electronic device (e.g., the electronic device 400 of FIG. 4) according to one or more embodiments may obtain first data related to a first biosignal corresponding to electrical characteristics, second data related to a second biosignal corresponding to optical characteristics, and third data corresponding to a temperature of skin.

The electronic device 400 according to one or more embodiments may obtain a first biosignal, a second biosignal, and a third biosignal related to temperature from an electrical sensor (e.g., the second electrical sensor 430 of FIG. 4), an optical sensor (e.g., the second optical sensor 440 of FIG. 4), and a temperature sensor (e.g., the second temperature sensor 450 of FIG. 4), respectively.

The electronic device 400 according to one or more embodiments may receive first data related to a first biosignal corresponding to electrical characteristics, second data related to a second biosignal corresponding to optical characteristics, and third data corresponding to a temperature of skin through the second communication circuit 420. The electronic device 400 according to one or more embodiments may receive the first data, the second data, and/or the third data from an external device (e.g., the wearable device 200 of FIG. 2A) through the second communication circuit 420.

The electronic device 400 according to one or more embodiments may obtain the first data, the second data, and/or the third data simultaneously or sequentially in operation 510, and the order of obtaining each signal may be changed in various ways.

The electronic device 400 according to one or more embodiments may identify whether the temperature of skin changes due to internal factors of the body in operation 520. For example, the temperature of skin may change due to internal factors of the body such as exercise or psychological state, and may also change due to external factors of the body according to changes in the external environment. The electronic device 400 according to one or more embodiments may identify whether it changes due to internal factors of the body or due to external factors of the body.

The electronic device 400 according to one or more embodiments may determine an exercise state of the user through heart rate (HR) or acceleration, and identify whether the temperature of skin changes due to internal factors of the body based on the determined exercise state.

The electronic device 400 according to one or more embodiments may determine whether the user is positioned outdoors based on location data (e.g., GPS signal), and obtain external environment data (e.g., temperature or humidity) corresponding to the user's location data. The electronic device 400 according to one or more embodiments may determine whether the user is positioned indoors based on location data (e.g., GPS signal), and obtain environment data (e.g., temperature or humidity) from an external device positioned indoors. The electronic device 400 according to one or more embodiments may include a separate sensor that measures external environment data (e.g., temperature or humidity). The electronic device 400 according to one or more embodiments may identify whether the temperature of skin changes due to internal factors of the body based on the obtained external environment data.

The electronic device 400 according to one or more embodiments may obtain the user's core temperature from an external device. The electronic device 400 according to one or more embodiments may estimate the user's core temperature based on first data related to the first biosignal, second data related to the second biosignal, and/or third data corresponding to the temperature of skin. The electronic device 400 according to one or more embodiments may identify whether the temperature of skin changes due to internal factors of the body based on the user's core temperature.

The electronic device 400 according to one or more embodiments may identify whether the temperature of skin changes due to internal factors of the body after identifying whether the temperature of skin changes. For example, the electronic device 400 may identify whether the temperature of skin changes due to internal factors of the body when the temperature of skin changes by more than a designated temperature (e.g., 0.5 degrees).

The electronic device 400 according to one or more embodiments may correct the first data and the second data with a designated third weight in operation 525 based on a result of identifying whether the temperature of skin changes due to internal factors of the body.

The electronic device 400 according to one or more embodiments may not apply the designated third weight when the temperature of skin changes due to internal factors of the body (operation 520-Yes).

The electronic device 400 according to one or more embodiments may apply the designated third weight to the first data and the second data in operation 525 when the temperature of skin does not change due to internal factors of the body (operation 520-No).

In one or more embodiments, when the temperature of skin changes due to internal factors of the body, blood flow volume and/or physical property changes of blood may occur relatively largely. Conversely, when the temperature of skin changes due to external factors of the body, blood flow volume and/or physical property changes of blood may occur relatively small. For example, the designated third weight may be designated as a value smaller than 1.

The electronic device 400 according to one or more embodiments may correct the first data and the second data based on the third data in operation 530. The electronic device 400 according to one or more embodiments may correct the first data and the second data respectively based on the temperature of skin.

With further reference to FIG. 6, when the user performs exercise (e.g., internal factors of the body), body water may decrease compared to before exercise, as the temperature of the body increases. However, a large error 600 occurs between changes in body water calculated respectively when temperature correction is performed and when temperature correction is not performed.

In particular, as illustrated in FIG. 6, when temperature correction is not performed, compared to when temperature correction is performed, changes in body water may not be properly reflected and errors may occur. The electronic device 400 according to one or more embodiments may quickly and accurately calculate changes in body water by detecting changes in body water based on biometric data corrected based on temperature.

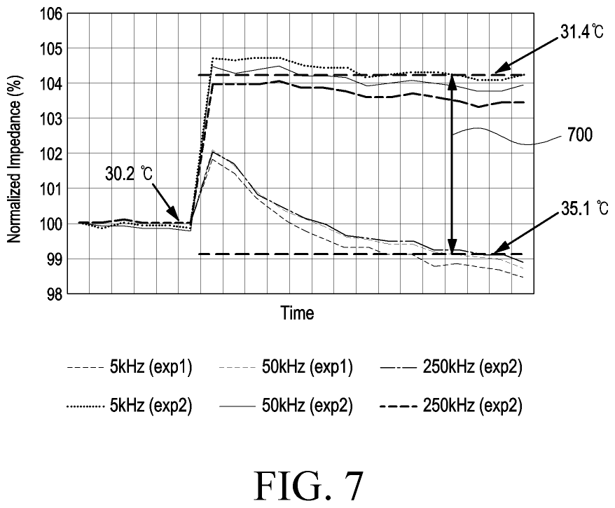

FIG. 7 illustrates a graph in which impedance of a plurality of frequencies changes according to a change in body temperature according to one or more embodiments of the disclosure.

With further reference to FIG. 7, a difference 700 may occur in the magnitude of impedance corresponding to electrical characteristics between when body temperature changes to a relatively high temperature (35.1° C.) and when it changes to a relatively low temperature (31.4° C.). A phenomenon in which impedance corresponding to a plurality of frequencies (5 kHz, 50 kHz, and 250 kHz) changes in response to changes in body temperature may be identified. This phenomenon may be caused by changes in blood flow volume according to temperature changes and/or changes in physical properties of blood according to temperature changes.

The impedance corresponding to each frequency in FIG. 7 has a difference 700 in the magnitude of impedance according to changes in body temperature as illustrated in [Table 1] below.

In one or more embodiments, the electronic device 400 may correct the measured impedance according to the temperature of skin. In one or more embodiments, the electronic device 400 may correct first data related to a first biosignal corresponding to electrical characteristics by applying a designated first weight. Here, the designated first weight is designated corresponding to the first data and may be designated to change according to the temperature of skin.

TABLE 1

| | High temperature | Low temperature | Impedance |
|---|---|---|---|
| Frequency | (35.1° C.) | (31.4° C.) | difference |
| 5 kHz | −1.53% | 4.37% | 5.90% |
| 50 kHz | −1.27% | 4.14% | 5.42% |
| 250 kHz | −1.10% | 3.43% | 4.53% |

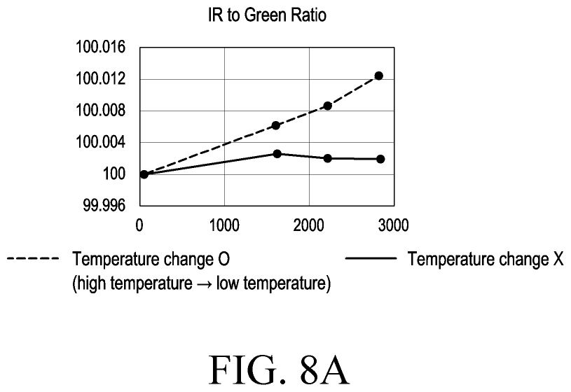

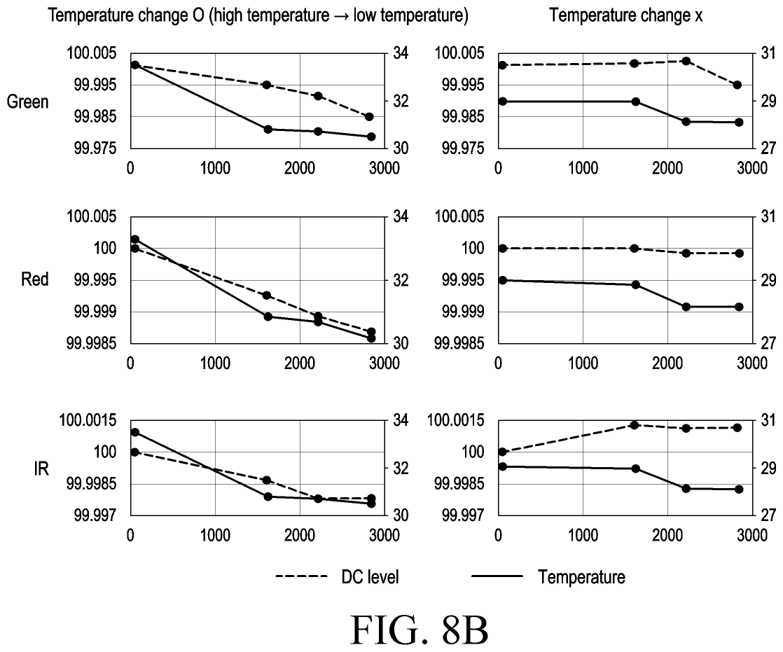

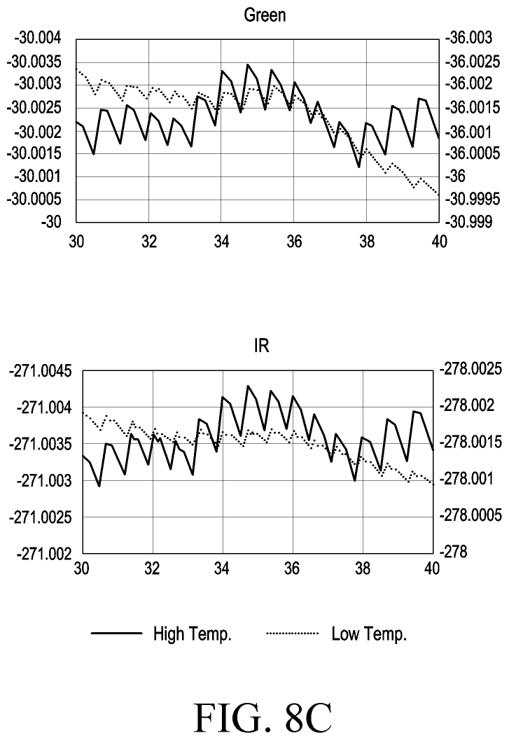

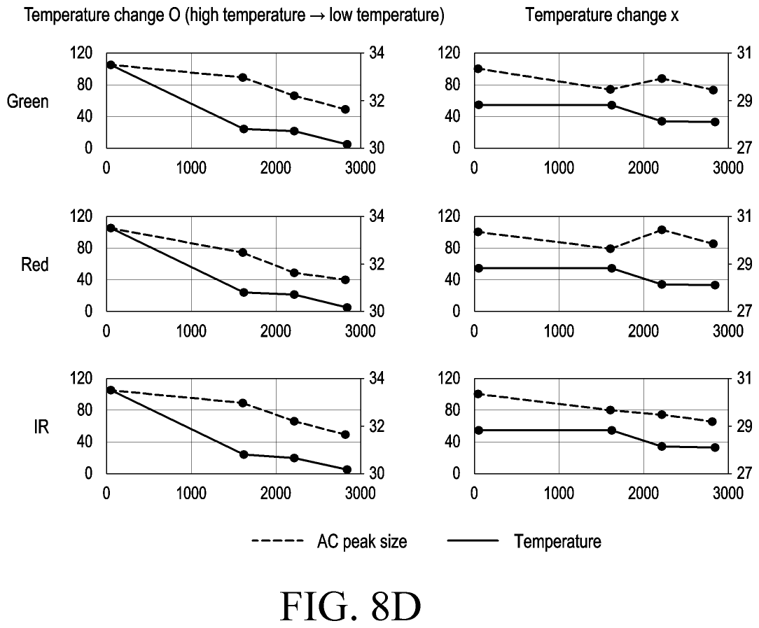

FIG. 8A is a graph illustrating a change in a ratio of green light to infrared light of PPG corresponding to whether temperature change occurs according to one or more embodiments of the disclosure. FIG. 8B is a graph illustrating a change in PPG DC level corresponding to whether temperature change occurs according to one or more embodiments of the disclosure. FIG. 8C is a graph illustrating PPG waveforms corresponding to respective temperatures according to one or more embodiments of the disclosure. FIG. 8D is a graph illustrating a change in PPG AC peak size corresponding to whether temperature change occurs according to one or more embodiments of the disclosure.

With further reference to FIG. 8A to FIG. 8D, compared to when no temperature change occurs, a phenomenon may be identified wherein data related to PPG corresponding to optical characteristics changes differently when temperature change occurs.

Specifically, as illustrated in FIG. 8A, compared to when no temperature change occurs (temperature change X), when a temperature changes from high temperature to low temperature occurs (temperature change O), a phenomenon in which the ratio of green light to infrared light (IR) gradually increases over time may be identified. For example, when a temperature changes from high temperature to low temperature occurs (temperature change O), a phenomenon in which the ratio of green light to infrared light (IR) relatively increases may be identified.

As illustrated in FIG. 8B, when no temperature change occurs (temperature change X), the PPG DC level may be maintained almost constant. In contrast, when a temperature changes from high temperature to low temperature occurs (temperature change O), a phenomenon in which the PPG DC level gradually decreases over time may be identified.

As illustrated in FIG. 8C, green light and infrared light (IR) may exhibit different PPG waveforms at relatively low temperatures (Low Temp.) and relatively high temperatures (High Temp). Specifically, in the case of green light, it may exhibit a relatively small change range at relatively low temperatures (Low Temp.) and may exhibit an aspect in which the magnitude gradually decreases over time. Further, green light may exhibit a relatively large change range at relatively high temperatures (High Temp) and may exhibit an aspect in which the magnitude is maintained over time.

In the case of infrared light (IR), it may exhibit a relatively small change range at relatively low temperatures (Low Temp.) and may exhibit an aspect in which the magnitude gradually decreases over time. Further, infrared light (IR) may exhibit a relatively large change range at relatively high temperatures (High Temp) and may exhibit an aspect in which the magnitude is maintained over time.

As illustrated in FIG. 8D, when no temperature change occurs (temperature change X), the PPG AC peak size may be maintained almost constant. In contrast, when a temperature changes from high temperature to low temperature occurs (temperature change O), a phenomenon in which the PPG AC peak size gradually decreases over time may be identified.

In one or more embodiments, the electronic device 400 may correct the measured PPG or data related to PPG according to the temperature of skin. In one or more embodiments, the electronic device 400 may correct second data related to a second biosignal corresponding to optical characteristics by applying a designated second weight. Here, the designated second weight is designated corresponding to the second data and may be designated to change according to the temperature of skin.

In one or more embodiments, the designated first weight and second weight are designated corresponding to the first data and the second data respectively, and may be designated to increase or decrease respectively according to the temperature of skin. For example, the designated first weight and second weight may be mapped to a table or graph corresponding to changes in the temperature of skin.

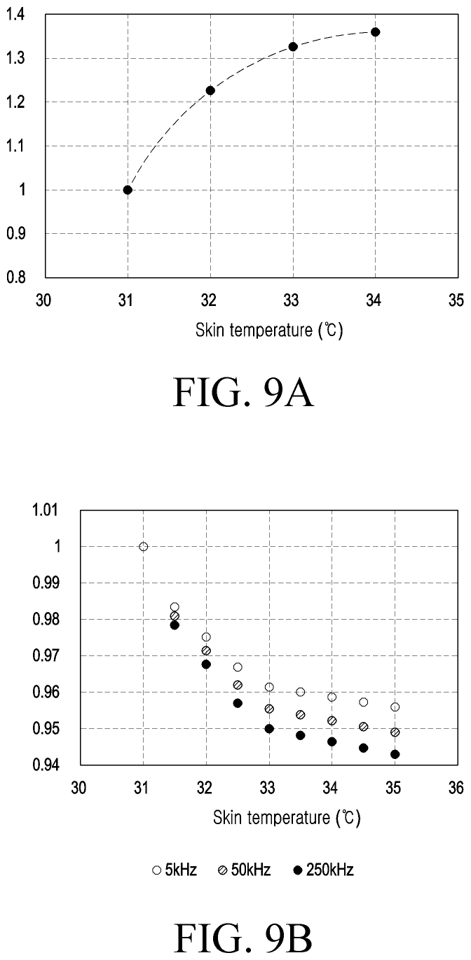

FIG. 9A illustrates a graph of a first weight designated according to a temperature of skin according to one or more embodiments of the disclosure. FIG. 9B illustrates a graph of a second weight designated according to a temperature of skin according to one or more embodiments of the disclosure.

TABLE 2

| Skin temperature (° C.) | Designated first weight |
|---|---|
| . . . | . . . |
| 31 | 1 |
| 32 | 1.21 |
| 33 | 1.33 |
| 34 | 1.36 |
| . . . | . . . |

TABLE 3

| Skin temperature | Designated second weight | | |
|---|---|---|---|
| (° C.) | 5 kHz | 50 kHz | 250 kHz |
| . . . | . . . | . . . | . . . |
| 31 | 1 | 1 | 1 |
| 31.5 | 0.9785 | 0.981 | 0.984 |
| 32 | 0.968 | 0.972 | 0.975 |
| 32.5 | 0.957 | 0.962 | 0.967 |
| . . . | . . . | . . . | . . . |
| 34 | 0.947 | 0.952 | 0.959 |
| 34.5 | 0.945 | 0.951 | 0.957 |
| 35 | 0.943 | 0.949 | 0.956 |
| . . . | . . . | . . . | . . . |

With further reference to FIG. 9A and Table 2, in one or more embodiments, the designated first weight may be designated to increase in magnitude based on an increase in the temperature of skin. For example, the designated first weight may be designated as 1 at a reference temperature (e.g., 31° C.) and may be designated to be greater than 1 at a temperature increased from the reference temperature. In one or more embodiments, the designated first weight may be designated corresponding to first data related to a first biosignal corresponding to electrical characteristics respectively.

The electronic device 400 according to one or more embodiments may correct first data related to a first biosignal corresponding to electrical characteristics (e.g., magnitude of impedance) that decreases as the temperature of skin increases by applying the designated first weight that is designated to increase as the temperature of skin increases.

With further reference to FIG. 9B and Table 3, in one or more embodiments, the designated second weight may be designated to decrease in magnitude based on an increase in the temperature of skin. For example, the designated second weight may be designated as 1 at a reference temperature (e.g., 31° C.) and may be designated to be smaller than 1 at a temperature increased from the reference temperature. In one or more embodiments, the designated second weight may be designated corresponding to second data related to a second biosignal corresponding to optical characteristics respectively.

The electronic device 400 according to one or more embodiments may correct second data related to a second biosignal corresponding to optical characteristics (e.g., ratio of green light to infrared light of PPG, PPG DC level, PPG AC peak size) that increases as the temperature of skin increases by applying the designated second weight that is designated to decrease as the temperature of skin increases.

Referring back to FIG. 5, the electronic device 400 according to one or more embodiments may calculate data related to a body water change of a body based on changes in the corrected first data and the corrected second data in operation 550.

The electronic device 400 according to one or more embodiments may calculate a change in the corrected first data. In one or more embodiments, the change in the corrected first data may be a current value (Corrected_Electrical_current) of the corrected first data compared to a previous value (Corrected_Electrical_previous) of the corrected first data as in the following equation (Equation 1). Alternatively, the change in the corrected first data may be calculated as a reciprocal of the current value (Corrected_Electrical_current) of the corrected first data compared to the previous value (Corrected_Electrical_previous) of the corrected first data as in the following equation (Equation 2).

$$ElectricalFeatureChange = {CorrectedElectrical}_{current} / {CorrectedElectrical}_{previous} \qquad [ Equation 1 ]$$

$$ElectricalFeatureChange = 1 / ( {CorrectedElectrical}_{current} / {CorrectedElectrical}_{previous} ) \qquad [ Equation 2 ]$$

The electronic device 400 according to one or more embodiments may calculate a change in the corrected second data. In one or more embodiments, the change in the corrected second data may be a current value (Corrected_Optical_current) of the corrected second data compared to a previous value (Corrected_Optical_previous) of the corrected second data as in the following equation (Equation 3). Alternatively, the change in the corrected first data may be calculated as a reciprocal of the current value of the corrected first data (Corrected_Optical_current) compared to the previous value (Corrected_Optical_previous) of the corrected second data as in the following equation (Equation 4).

$$OpticalFeatureChange = {CorrectedOptical}_{current} / {CorrectedOptical}_{previous} \qquad [ Equation 3 ]$$

$$OpticalFeatureChange = 1 / ( {CorrectedOptical}_{current} / {CorrectedOptical}_{previous} ) \qquad [ Equation 4 ]$$

The electronic device 400 according to one or more embodiments may calculate data related to body water change by applying a designated fifth weight and a designated sixth weight to the change in the corrected first data and the change in the corrected second data respectively.

In one or more embodiments, the electronic device 400 may calculate a body water change (Body Water Change) of the body based on a sum of a change in the corrected first data (Electrical Feature Change) to which the designated fifth weight (Weight 5) is applied and a change in the corrected second data (Optical Feature Change) to which the designated sixth weight (Weight 6) is applied as in the following equation (Equation 5).

$$BodyWaterChange = Weight 5 \times ElectricalFeatureChange + Weight 6 \times OpticalFeatureChange \qquad [ Equation 5 ]$$

In one or more embodiments, the sum of the designated fifth weight and the designated sixth weight may be maintained constant. For example, the sum of the designated fifth weight and the designated sixth weight may be maintained constant at 1.

In one or more embodiments, the designated fifth weight and the designated sixth weight may be changed based on a change in the temperature of skin. For example, the designated fifth weight and the designated sixth weight may be designated according to the temperature of skin as illustrated in Table 4 below.

TABLE 4

| Skin temperature | Designated fifth | Designated sixth |
|---|---|---|
| (° C.) | weight | weight |
| . . . | . . . | . . . |
| 31 | 0.5 | 0.5 |
| 32 | 0.55 | 0.45 |
| 33 | 0.60 | 0.4 |
| 34 | 0.65 | 0.35 |
| . . . | . . . | . . . |

In one or more embodiments, the designated fifth weight may be designated to gradually increase as the temperature of skin increases. In one or more embodiments, the designated sixth weight may be designated to gradually decrease as the temperature of skin increases.

For example, the temperature of skin often increases as the user performs exercise, and first data related to electrical characteristics may be relatively robust to motion artifact. In one or more embodiments, the magnitude of the designated fifth weight corresponding to first data related to electrical characteristics may be designated to be large.

Further, in the case of body water decrease (dehydration), since the reliability of first data related to electrical characteristics and second data related to optical characteristics is similar, the designated fifth weight and the designated sixth weight may be designated at similar levels.

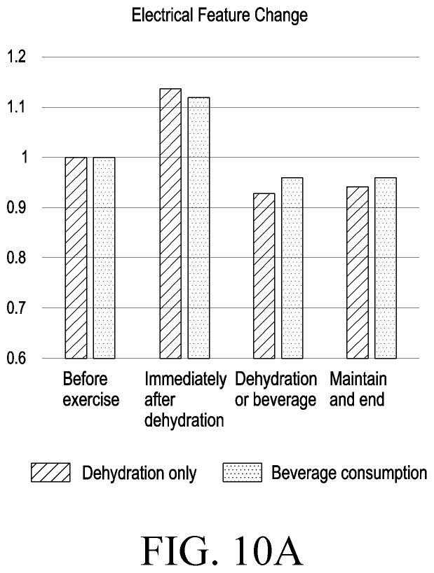

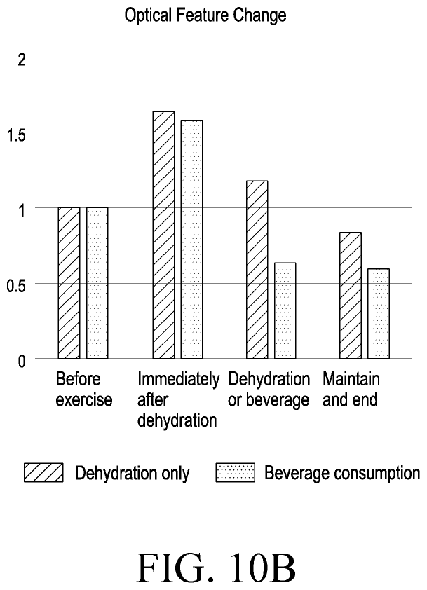

FIG. 10A is a graph illustrating a change in first data corresponding to respective circumstances of a user according to one or more embodiments of the disclosure. FIG. 10B is a graph illustrating a change in second data corresponding to respective circumstances of a user according to one or more embodiments of the disclosure.

With further reference to FIG. 10A, looking at the change in first data related to electrical characteristics when dehydration occurs due to user's exercise, almost no difference in the magnitude of change in first data occurs between when no beverage is consumed after dehydration (dehydration only) and when water is consumed after dehydration (beverage consumption). When water is consumed after dehydration (beverage consumption), it is expected that there is a difference in body water change of the body compared to when no beverage is consumed after dehydration (dehydration only). However, in the case of change in first data related to electrical characteristics, it may have a relatively small effect on the change of body water in response to water consumption.

With further reference to FIG. 10B, looking at the change in second data related to optical characteristics when dehydration occurs due to user's exercise, a distinct difference in the magnitude of change in second data occurs between when no beverage is consumed after dehydration (dehydration only) and when water is consumed after dehydration (beverage consumption). In particular, after dehydration due to user's exercise, the change in second data exhibits a distinct difference when water is consumed (beverage consumption) compared to when no beverage is consumed (dehydration only). In the case of change in second data related to optical characteristics, it may have a relatively large effect on the change of body water in response to water consumption.

In other words, in the case of body water increase (e.g., water re-consumption), the reliability of change in second data related to optical characteristics may be relatively high. In one or more embodiments, the magnitude of the designated sixth weight corresponding to second data related to optical characteristics may be designated to be relatively large.

In one or more embodiments, the designated fifth weight and the designated sixth weight may be designated according to body water change rate as illustrated in Table 5 below.

TABLE 5

| Body water change | Designated fifth | Designated sixth |
|---|---|---|
| rate (%) | weight | weight |
| . . . | . . . | . . . |
| +0.3% | 0.44 | 0.56 |
| +0.2% | 0.46 | 0.54 |
| +0.1% | 0.48 | 0.52 |
| 0 | 0.5 | 0.5 |
| −0.1% | 0.5 | 0.5 |
| . . . | 0.5 | 0.5 |

In one or more embodiments, when there is no change in body water change rate or when body water decreases (e.g., dehydration), the designated fifth weight and the designated sixth weight may be designated to be maintained constant. In one or more embodiments, when body water increases (e.g., hydration), the designated fifth weight may be designated to increase and the designated sixth weight may be designated to decrease. In one or more embodiments, as the magnitude of the body water change rate increases, the designated fifth weight may be designated to gradually increase and the designated sixth weight may be designated to gradually decrease.

Referring back to FIG. 5, the electronic device 400 according to one or more embodiments may transmit data related to the generated body water change rate to an external device (e.g., the wearable device 200 of FIG. 2A) through a communication circuit in operation 570.

The electronic device 400 according to one or more embodiments may output feedback to an output module (e.g., the second output module 460 of FIG. 4) based on data related to the generated body water change rate in operation 570.

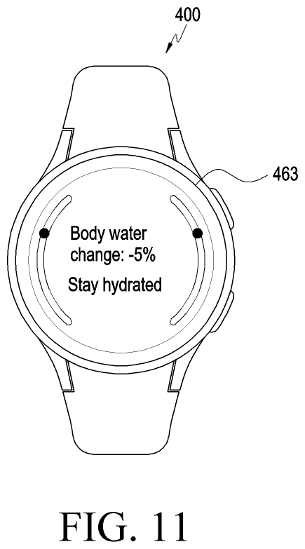

FIG. 11 illustrates a second display 463 of an electronic device 400 providing visual feedback according to one or more embodiments of the disclosure.

Referring to FIG. 11, the electronic device 400 according to one or more embodiments may output feedback to an output module (e.g., the second output module 460 of FIG. 4) based on data related to the generated body water change rate.

The electronic device 400 according to one or more embodiments may output visual, auditory, and/or tactile feedback to the second output module 460 to the user. In one or more embodiments, the electronic device 400 may output feedback related to the body water change rate based on data related to the generated body water change rate. In one or more embodiments, the electronic device 400 may provide visual feedback to the second display 463, provide auditory feedback to the second speaker 465, or provide tactile feedback to the second haptic module 467.

The electronic device 400 according to one or more embodiments may provide visual feedback to the second display 463 based on data related to the body water change rate as illustrated in FIG. 11. In one or more embodiments, the electronic device 400 may visually display data related to the generated body water change rate on the second display 463.

In one or more embodiments, the electronic device 400 may provide feedback to the second output module 460 when the body water change rate falls outside a designated range (e.g., −4% to +4% range) based on data related to the generated body water change rate.

A wearable device 200; 101 according to one or more embodiments of the disclosure may include a communication circuit 220; 190 configured to transmit or receive data or a signal with an external electronic device E, an output module 260, an electrical sensor 230 measuring a first biosignal related to an electrical characteristic, an optical sensor 240 measuring a second biosignal related to an optical characteristic, a temperature sensor 250 measuring a temperature of skin, and at least one processor 210; 120 operatively connected to the communication circuit 220; 190, the output module 260, the electrical sensor 230, the optical sensor 240, and the temperature sensor 250. The at least one processor 210; 120 may be configured to obtain, from the electrical sensor 230, the optical sensor 240, and the temperature sensor 250, respectively, the first biosignal, the second biosignal, and a third biosignal related to the temperature. The at least one processor 210; 120 may be configured to transmit, through the communication circuit 220; 190, first data related to the obtained first biosignal, second data related to the obtained second biosignal, and third data related to the third biosignal to the external electronic device E. The at least one processor 210; 120 may be configured to receive, through the communication circuit 220; 190, data related to a body water change of a body calculated based on changes in the first data and the second data respectively corrected by the third data from the external electronic device E. The at least one processor 210; 120 may be configured to output feedback related to the body water change to the output module 260 based on the received data related to the body water change.

In the wearable device 200; 101 according to one or more embodiments, the output module 260 may be configured to include at least one of a display 263 providing visual feedback, a speaker 265 providing auditory feedback, or a haptic module 267 providing tactile feedback.

In the wearable device 200; 101 according to one or more embodiments, the electrical sensor 230 may be measuring impedance of the skin corresponding to a specific frequency. The first data related to the first biosignal may include data related to at least one of a magnitude of impedance, a phase of impedance, a resistance, a reactance, or a ratio of impedance at different frequencies.

In the wearable device 200; 101 according to one or more embodiments, the optical sensor 240 may be configured to optically measure the second biosignal related to blood flow. The second data related to the second biosignal may include data related to at least one of absorbance, photoplethysmography PPG AC peak, PPG DC level, ratio between PPG DC and PPG AC, ratio of PPG DC at different frequencies, or ratio of PPG AC at different frequencies.

A method of operating the wearable device 200; 101 according to one or more embodiments of the disclosure may include obtaining 310, from an electrical sensor 230, an optical sensor 240, and a temperature sensor 250, respectively, a first biosignal related to an electrical characteristic, a second biosignal related to an optical characteristic, and a third biosignal related to temperature. The method of operating the wearable device 200; 101 according to one or more embodiments may include transmitting 330, through a communication circuit 220; 190, first data related to the obtained first biosignal, second data related to the obtained second biosignal, and third data related to the third biosignal to an external electronic device E. The method of operating the wearable device 200; 101 according to one or more embodiments may include receiving 350, through the communication circuit 220; 190, data related to a body water change of a body calculated based on changes in the first data and the second data respectively corrected by the third data from the external electronic device E. The method of operating the wearable device 200; 101 according to one or more embodiments may include outputting 370 feedback related to the body water change to an output module 260 based on the received data related to the body water change.

A non-transitory, computer-readable storage medium storing one or more programs according to one or more embodiments of the disclosure may include obtaining 310, from an electrical sensor 230, an optical sensor 240, and a temperature sensor 250, respectively, a first biosignal related to an electrical characteristic, a second biosignal related to an optical characteristic, and a third biosignal related to temperature, based on execution of an application. The storage medium according to one or more embodiments may include transmitting 330, through a communication circuit 220; 190, first data related to the obtained first biosignal, second data related to the obtained second biosignal, and third data related to the third biosignal to an external electronic device E. The storage medium according to one or more embodiments may include receiving 350, through the communication circuit 220; 190, data related to a body water change of a body calculated based on changes in the first data and the second data respectively corrected by the third data from the external electronic device E. The storage medium according to one or more embodiments may include outputting 370 feedback related to the body water change to an output module 260 based on the received data related to the body water change.

An electronic device 400; 101 according to one or more embodiments of the disclosure may include a communication circuit 420; 190 configured to transmit or receive data or a signal with an external device 200, an output module, and at least one processor 410; 120 operatively connected to the communication circuit 420; 190 and the output module 460. The at least one processor 410; 120 may be configured to obtain first data related to a first biosignal corresponding to an electrical characteristic, second data related to a second biosignal corresponding to an optical characteristic, and third data corresponding to a temperature of skin. The at least one processor 410; 120 may be configured to correct the first data and the second data based on the third data. The at least one processor 410; 120 may be configured to calculate data related to a body water change of a body based on changes in the corrected first data and the corrected second data. The at least one processor 410; 120 may be configured to transmit, through the communication circuit 420; 190, the calculated data related to the body water change to the external device 200, or output feedback to the output module 460 based on the calculated data related to the body water change.

In the electronic device 400; 101 according to one or more embodiments, the at least one processor 410; 120 may be configured to receive, through the communication circuit 420; 190, the first data, the second data, and the third data from the external device 200 as at least a portion of obtaining the first data, the second data, and the third data.

The electronic device 400; 101 according to one or more embodiments may further include an electrical sensor 430 measuring the first biosignal, an optical sensor 440 measuring the second biosignal, and a temperature sensor 450 measuring the temperature. The at least one processor 410; 120 may be configured to obtain, from the electrical sensor 430, the optical sensor 440, and the temperature sensor 450, respectively, the first data, the second data, and the third data, as at least a portion of obtaining the first data, the second data, and the third data.

In the electronic device 400; 101 according to one or more embodiments, the at least one processor 410; 120 may be configured to correct the first data and the second data based on a designated first weight and a designated second weight, respectively, as at least a portion of correcting the first data and the second data. The designated first weight may be designated to change according to the temperature of the skin corresponding to the first data. The designated second weight may be designated to change according to the temperature of the skin corresponding to the second data.

In the electronic device 400; 101 according to one or more embodiments, the designated first weight may be designated to increase in magnitude based on an increase in the temperature of the skin. The designated second weight may be designated to decrease in magnitude based on the increase in the temperature of the skin.

In the electronic device 400; 101 according to one or more embodiments, the at least one processor 410; 120 may be configured to identify whether the temperature of the skin changes due to an internal factor of the body. The at least one processor 410; 120 may be configured to correct the first data and the second data with a designated third weight based on the identified result.

In the electronic device 400; 101 according to one or more embodiments, the at least one processor 410; 120 may be configured to calculate the data related to the body water change based on a sum of a change in the corrected first data to which a designated fifth weight is applied and a change in the corrected second data to which a designated sixth weight is applied, as at least a portion of calculating the data related to the body water change of the body. A sum of the designated fifth weight and the designated sixth weight may be constant.

In the electronic device 400; 101 according to one or more embodiments, the designated fifth weight and the designated sixth weight may be changed based on a change in the temperature of the skin.

In the electronic device 400; 101 according to one or more embodiments, the designated fifth weight and the designated sixth weight may be changed based on the body water change.

A method of operating an electronic device 400; 101 according to one or more embodiments of the disclosure may include obtaining 510 first data related to a first biosignal corresponding to an electrical characteristic, second data related to a second biosignal corresponding to an optical characteristic, and third data related to a temperature of skin. The method of operating the electronic device 400; 101 according to one or more embodiments may include correcting 530 the first data and the second data based on the third data. The method of operating the electronic device 400; 101 according to one or more embodiments may include calculating 550 data related to a body water change of a body based on changes in the corrected first data and the corrected second data. The method of operating the electronic device 400; 101 according to one or more embodiments may include transmitting, through a communication circuit 420; 190, the calculated data related to the body water change to an external device 200, or outputting 570 feedback to an output module 460 based on the calculated data related to the body water change.

In the method of operating the electronic device 400; 101 according to one or more embodiments, the obtaining 510 the first data, the second data, and the third data may receive, through the communication circuit 420; 190, the first data, the second data, and the third data from the external device 200.

In the method of operating the electronic device 400; 101 according to one or more embodiments, the obtaining 510 the first data, the second data, and the third data may receive, from an electrical sensor 430 measuring the first biosignal, an optical sensor 440 measuring the second biosignal, and a temperature sensor 450 measuring the temperature, respectively, the first biosignal, the second biosignal, and the third biosignal.

In the method of operating the electronic device 400; 101 according to one or more embodiments, the correcting 530 the first data and the second data may correct the first data and the second data based on a designated first weight and a designated second weight, respectively. The designated first weight may be designated to change according to the temperature of the skin corresponding to the first data. The designated second weight may be designated to change according to the temperature of the skin corresponding to the second data.

The method of operating the electronic device 400; 101 according to one or more embodiments may further include identifying 520 whether the temperature of the skin changes due to an internal factor of the body. The method of operating the electronic device 400; 101 according to one or more embodiments may further include correcting 525 the first data and the second data with a designated third weight based on the identified result.

In the method of operating the electronic device 400; 101 according to one or more embodiments, the calculating 550 the data related to the body water change of the body may calculate the data related to the body water change based on a sum of a change in the corrected first data to which a designated fifth weight is applied and a change in the corrected second data to which a designated sixth weight is applied. A sum of the designated fifth weight and the designated sixth weight may be constant.

In the method of operating the electronic device 400; 101 according to one or more embodiments, the designated fifth weight and the designated sixth weight may be changed based on at least one of a change in the temperature of the skin or the body water change.

A non-transitory, computer-readable storage medium storing one or more programs according to one or more embodiments of the disclosure may include obtaining 510 first data related to a first biosignal corresponding to an electrical characteristic, second data related to a second biosignal corresponding to an optical characteristic, and third data related to a temperature of skin, based on execution of an application. The storage medium according to one or more embodiments may include correcting 530 the first data and the second data based on the third data. The storage medium according to one or more embodiments may include calculating 550 data related to a body water change of a body based on changes in the corrected first data and the corrected second data. The storage medium according to one or more embodiments may include transmitting, through a communication circuit 420; 190, the calculated data related to the body water change to an external device 200, or outputting 570 feedback to an output module 460 based on the calculated data related to the body water change.

In a storage medium storing computer-readable instructions according to one or more embodiments of the disclosure, wherein the instructions, when executed by at least one processor 210; 120 of a wearable device 200; 101, cause the wearable device 200; 101 to obtain, from an electrical sensor 230 measuring a first biosignal related to an electrical characteristic, an optical sensor 240 measuring a second biosignal related to an optical characteristic, and a temperature sensor 250 measuring a temperature of skin, respectively, the first biosignal, the second biosignal, and a third biosignal related to the temperature.

The instructions according to one or more embodiments of the disclosure, may cause to transmit, through a communication circuit 220; 190 configured to transmit or receive data or a signal with an external electronic device E, first data related to the obtained first biosignal, second data related to the obtained second biosignal, and third data related to the third biosignal to the external electronic device E.

The instructions according to one or more embodiments of the disclosure, may cause to receive through the communication circuit 220; 190, data related to a body water change of a body calculated based on changes in the first data and the second data respectively corrected by the third data from the external electronic device E.

The instructions according to one or more embodiments of the disclosure, may cause to output feedback related to the body water change to an output module 260, based on the received data related to the body water change.

The electronic device according to an embodiment may be one of various types of electronic devices. The electronic devices may include, for example, a portable communication device (e.g., a smart phone), a computer device, a portable multimedia device, a portable medical device, a camera, an electronic device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

An embodiment of the disclosure may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The storage medium readable by the machine may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities. Some of the plurality of entities may be separately disposed in different components. According to an embodiment, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

What is claimed is:

1. A wearable device comprising:
a communication circuit configured to transmit data or a signal to an external electronic device or receive data or a signal from the external device;
an output device;
an electrical sensor configured to measure a first biosignal related to an electrical characteristic;
an optical sensor configured to measure a second biosignal related to an optical characteristic;
a temperature sensor configured to measure a temperature;
at least one processor; and
memory storing instructions,
wherein the instructions, when executed by the at least one processor individually or collectively, cause the wearable device to:
obtain the first biosignal from the electrical sensor, the second biosignal from the optical sensor, and a third biosignal related to the temperature from the temperature sensor;
transmit, through the communication circuit, first data related to the obtained first biosignal, second data related to the obtained second biosignal, and third data related to the third biosignal to the external electronic device;
receive, through the communication circuit, data related to a body water change of a body determined based on changes in the first data and the second data respectively corrected by the third data from the external electronic device; and
output feedback related to the body water change to the output device based on the received data related to the body water change.

2. The wearable device of claim 1, wherein the output device comprises at least one of a display configured to provide visual feedback, a speaker configured to provide auditory feedback, or a haptic module configured to provide tactile feedback.

3. The wearable device of claim 1, wherein the electrical sensor is configured to measure impedance of skin corresponding to a specific frequency, and
wherein the first data related to the first biosignal includes data related to at least one of a magnitude of impedance, a phase of impedance, a resistance, a reactance, or a ratio of impedance at different frequencies.

4. The wearable device of claim 1, wherein the optical sensor is configured to optically measure the second biosignal related to blood flow, and
wherein the second data related to the second biosignal includes data related to at least one of an absorbance, a photoplethysmography (PPG) alternating current (AC) peak, a PPG direct current (DC) level, a ratio between PPG DC and PPG AC, a ratio of PPG DC at different frequencies, or a ratio of PPG AC at different frequencies.

5. An electronic device comprising:
a communication circuit configured to transmit data or a signal to an external device or receive data or a signal from the external device;
an output device;
at least one processor; and
memory storing instructions,
wherein the instructions, when executed by the at least one processor individually or collectively, cause the electronic device to:
obtain first data related to a first biosignal corresponding to an electrical characteristic, second data related to a second biosignal corresponding to an optical characteristic, and third data corresponding to a temperature;
correct the first data and the second data based on the third data;
determine data related to a body water change of a body based on changes in the corrected first data and the corrected second data; and
transmit, through the communication circuit, the data related to the body water change, to the external device, or output feedback to the output device based on the data related to the body water change.

6. The electronic device of claim 5, wherein the instructions, when executed by the at least one processor individually or collectively, cause the electronic device to receive, through the communication circuit, the first data, the second data, and the third data from the external device as at least a portion of obtaining the first data, the second data, and the third data.

7. The electronic device of claim 5, further comprising:
an electrical sensor configured to measure the first biosignal;
an optical sensor configured to measure the second biosignal; and
a temperature sensor configured to measure the temperature,
wherein the instructions, when executed by the at least one processor individually or collectively, cause the electronic device to obtain the first data from the electrical sensor, the second data from the optical sensor, and the third data from the temperature sensor as at least a portion of obtaining the first data, the second data, and the third data.

8. The electronic device of claim 5, wherein the instructions cause the electronic device to correct the first data and the second data based on a designated first weight and a designated second weight, respectively, as at least a portion of correcting the first data and the second data,
wherein the designated first weight is designated to change according to the temperature corresponding to the first data, and
wherein the designated second weight is designated to change according to the temperature corresponding to the second data.

9. The electronic device of claim 8, wherein the designated first weight is designated to increase in magnitude based on an increase in the temperature, and
wherein the designated second weight is designated to decrease in magnitude based on the increase in the temperature.

10. The electronic device of claim 5, wherein the instructions, when executed by the at least one processor individually or collectively, cause the electronic device to:
identify whether the temperature changes due to an internal factor of the body; and
correct the first data and the second data with a designated third weight based on a result of identifying whether the temperature changes.

11. The electronic device of claim 5, wherein the instructions, when executed by the at least one processor individually or collectively, cause the electronic device to determine the data related to the body water change based on a sum of a change in the corrected first data to which a designated fifth weight is applied, and a change in the corrected second data to which a designated sixth weight is applied, as at least a portion of determining the data related to the body water change, and
wherein a sum of the designated fifth weight and the designated sixth weight is constant.

12. The electronic device of claim 11, wherein the designated fifth weight and the designated sixth weight are changed based on a change in the temperature.

13. The electronic device of claim 11, wherein the designated fifth weight and the designated sixth weight are changed based on the body water change.

14. A method of operating an electronic device, the method comprising:
obtaining first data related to a first biosignal corresponding to an electrical characteristic, second data related to a second biosignal corresponding to an optical characteristic, and third data related to a temperature;
correcting the first data and the second data based on the third data;
determining data related to a body water change of a body based on changes in the corrected first data and the corrected second data; and
transmitting, through a communication circuit, the data related to the body water change to an external device, or outputting feedback to an output device based on the data related to the body water change.

15. The method of claim 14, wherein the obtaining the first data, the second data, and the third data comprises receiving, through the communication circuit, the first data, the second data, and the third data from the external device.

16. The method of claim 14, wherein the obtaining the first data, the second data, and the third data comprises receiving the first biosignal from an electrical sensor measuring the first biosignal, the second biosignal from an optical sensor measuring the second biosignal, and the third biosignal from a temperature sensor measuring the temperature.

17. The method of claim 14, wherein the correcting the first data and the second data comprises correcting the first data and the second data based on a designated first weight and a designated second weight, respectively,
wherein the method further comprises:
changing the designated first weight according to the temperature corresponding to the first data, and
changing the designated second weight according to the temperature corresponding to the second data.

18. The method of claim 14, further comprises:
identifying whether the temperature changes due to an internal factor of the body; and
correcting the first data and the second data with a designated third weight based on a result of the identifying.

19. The method of claim 14, wherein the determining the data related to the body water change of the body comprises determining the data related to the body water change based on a sum of a change in the corrected first data to which a designated fifth weight is applied, and a change in the corrected second data to which a designated sixth weight is applied, and
wherein a sum of the designated fifth weight and the designated sixth weight may be constant.

20. The method of claim 19, further comprising changing one or more of the designated fifth weight and the designated sixth weight based on at least one of a change in the temperature or the body water change.
